(19) United States
(12) Patent Application Publication
(10) Pub. No.: US 2026/0224558 A1
(43) Pub. Date: Aug. 6, 2026

(54) METHYLNALTREXONE COMPOSITION FOR ORAL ADMINISTRATION

(71) Applicant: Lupin Limited, Mumbai (IN)

(72) Inventors: Ashish Ashokrao Deshmukh, Pune (IN); Brijesh Purohit, Pune (IN); Atul Kaushik, Pune (IN); Makarand Krishnakumar Avachat, Pune (IN)

(21) Appl. No.: 19/529,933

(22) Filed: Feb. 4, 2026

(30) Foreign Application Priority Data
Feb. 4, 2025 (IN) 202521009192

(51) Int. Cl. A61K 31/485; A61K 9/00; A61K 9/28; A61K 31/225

(52) U.S. Cl. CPC A61K 31/485; A61K 9/0053; A61K 9/2893; A61K 31/225

(57) ABSTRACT

The present invention relates to a stable pharmaceutical composition comprising methylnaltrexone or its pharmaceutically acceptable salts. The invention provides an alternative methylnaltrexone composition for oral administration for the effective treatment of opioid-induced constipation (OIC). The present invention further relates to compositions comprising methylnaltrexone or its pharmaceutically acceptable salts and docusate sodium, as well as processes for their preparation. The compositions provide the desired immediate release of methylnaltrexone and exhibit stability under accelerated storage conditions. The compositions are administered orally once daily for the treatment of opioid-induced constipation in adult patients with cancer or chronic non-cancer pain. Additionally, the invention provides methylnaltrexone compositions for oral administration that are bioequivalent to the currently marketed Relistor® tablets.

### FIELD OF INVENTION

The present invention relates to solid oral pharmaceutical compositions of methylnaltrexone or a pharmaceutically acceptable salt thereof. The present invention also relates to a process of preparing and using said compositions for treatment of opioid-induced constipation (OIC) in adult patients with cancer and chronic non-cancer pain. More particularly, the invention relates to immediate release composition of methylnaltrexone or a pharmaceutically acceptable salt thereof and docusate sodium.

### BACKGROUND

Opioids are highly administered for the management of acute and chronic pain. Opioid usage for the management of pain in cancer and non-cancer patients is associated with several centrally and peripherally mediated adverse effects. Peripheral mu-opioid receptors on the gastrointestinal (GI) tract are responsible for opioid-induced bowel dysfunctions (OIBD). By affecting the transit time of the GI tract, opioids cause constipation. Constipation occurs in approximately 40-90% of individuals taking opioids for pain control. Extensive prevalence of opioid-induced constipation (OIC) experienced by patients is one of the major causes of discontinuing analgesic treatment. Therefore, non-compliance and avoiding opioid therapy may occur due to these adverse effects. Quality of life (QOL) is significantly impaired in patients with OIC. Both mental and physical components in these patients are affected. On the other hand, discontinuation of opioid treatment due to OIC results in uncontrolled pain that affects QOL.

Peripheral mu-opioid receptor antagonists (PAMORAs) are successful options in OIC treatment. Methylnaltrexone and its salts block the mu-opioid receptor in the GI tract, without affecting the central opioid-induced analgesia. It is the first PAMORA approved for the OIC in patients with advanced illness when laxatives were not efficient. Methylnaltrexone, a quaternary derivative of naltrexone, is a peripherally nonselective mu-opioid receptor antagonist which can provide causal treatment for opioid-induced constipation (OIC), yet does not interfere with the analgesic effect. Methylnaltrexone, characterized by the general structure of a quaternary ammonium salt, exhibits increased polarity and reduced lipid solubility. Therefore, methylnaltrexone has restricted access to the blood-brain barrier and decreases the constipating effects of opioid pain medications. However, as a hydrophilic compound, methylnaltrexone has limited gastrointestinal absorption (Yuan, C. S et al, 1997 and Becker G et al, 2007). Due to the positive charge of the quaternary amine, methylnaltrexone is poorly absorbed in the gastrointestinal tract. In general, on oral administration, less than 5% of methylnaltrexone is absorbed into the bloodstream. The structure of methylnaltrexone is shown below.

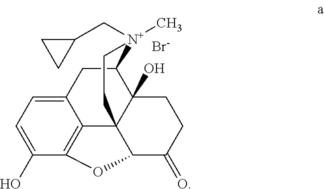 [SMILES: C[N+]1(CC2CC2)CC[C@]23c4c5ccc(O)c4O[C@H]2C(=O)CC[C@@]3(O)[C@H]1C5 ; [Br-]]

It has been used in patients to reduce opioid-induced side effects such as constipation, pruritus, nausea, and urinary retention (see, e.g., U.S. Pat. Nos. 5,972,954, 5,102,887, 4,861,781, and 4,719,215; and Yuan et al., Drug and Alcohol Dependence 1998, 52, 161). The dosage form of methylnaltrexone used most often in these studies has been a solution of methylnaltrexone for intravenous injections. Methylnaltrexone subcutaneous injection Relistor® is available for the treatment of opioid induced constipation in patients with advanced illness receiving palliative care when response to laxative therapy has not been sufficient. The dose of methylnaltrexone for treating methadone maintenance patients was explored in U.S. Pat. No. 6,559,158.

An oral dosage form that releases certain opioid antagonists such as naloxone, N-methylnaloxone, and N-methylnaltrexone “over the whole gastrointestinal tract is disclosed in U.S. Pat. No. 6,419,959.” Further it states that opioid antagonists are not always suitable for administration in an immediate release form due to dose limiting side effects. To address these issues, the U.S. Pat. No. 6,419,959 suggests dosing certain opioid antagonists, including methylnaltrexone, in a controlled-release dosage form, thereby delivering these antagonists at acceptable doses locally across the entire gastrointestinal tract. However, data with respect to methylnaltrexone specifically was not reported.

In U.S. Pat. No. 6,274,591, it was demonstrated that an enteric coated methylnaltrexone which released substantially no methylnaltrexone in the stomach was more effective than uncoated methylnaltrexone in antagonizing the oral-cecal delay caused by morphine. Hence, U.S. Pat. No. 6,274,591 suggests and claims delivering effective amounts of Methylnaltrexone using an oral dosage that bypasses the stomach altogether.

Immediate release methylnaltrexone is commercially available as Relistor® tablets 150 mg. The film-coated tablet contains 150 mg of methylnaltrexone bromide which is equivalent to 122.5 mg methylnaltrexone.

Patents and publications related to Relistor® tablets are U.S. Pat. Nos. 8,524,276, 8,956,651, 9,314,461, U.S. Ser. No. 10/307,417, U.S. Ser. No. 10/376,505, U.S. Pat. No. 1,037,650, U.S. Ser. No. 10/507,206, US 20160206612, US 20200121673 and US 20130317050, wherein an ion pair between the positively charged methylnaltrexone and a negatively charged moiety was postulated to make a “pair” that is more hydrophobic than methylnaltrexone bromide and thereby enhanced the absorption of Methylnaltrexone in the stomach.

Ion pairing was done using methylnaltrexone and an amphiphilic pharmaceutically acceptable excipient particularly, sodium lauryl sulfate in a solid dosage form, together with a rapid-acting disintegrant e.g., a carbon dioxide generating disintegrant which when dissolved in solution was effective to induce laxation. Inventors determined the octanol/water partition coefficient of the prepared ion pairs of methylnaltrexone with amphiphilic excipients and apparent octanol/water partition coefficient found at least 0.25 at pH between 1 and 4.

US 20040259899, disclosed the composition of opioid antagonists such as methylnaltrexone in combination with laxatives or stool softeners for the treatment of constipation. Further disclosed the enterically coated sustained release tablets of methylnaltrexone in combination with stool softener such as docusate sodium.

US 20220096461, disclosed the liquid oral compositions consisting of opioid antagonist such as methylnaltrexone and docusate by forming an ion pair of methylnaltrexone-docusate; and pharmacokinetics of the prepared oral liquid compositions was compared with Relistor® tablets.

The present invention provides alternate methylnaltrexone composition for oral administration for the effective treatment of OIC. Present invention also provides process for preparation of the said alternate methylnaltrexone composition for an oral administration for the treatment of OIC. Additionally, the present invention provides methylnaltrexone composition for oral administration that is bioequivalent with currently marketed Relistor® tablets.

### OBJECTIVE OF THE INVENTION

The present invention provides the following aspects, subject matters, and preferred embodiments, which respectively taken alone or in combination, contribute to solving the object of the present invention.

The main objective of the invention is to provide immediate release pharmaceutical compositions comprising methylnaltrexone or a pharmaceutically acceptable salt thereof, docusate and its pharmaceutically acceptable salt and a pharmaceutically acceptable excipient for the treatment of opioid-induced constipation (OIC).

It is another objective of the present invention to provide pharmaceutical compositions containing from about 50 mg to about 500 mg of methylnaltrexone bromide. Preferably the invention provides pharmaceutical compositions containing from about 150 mg to about 450 mg of methylnaltrexone bromide.

It is yet another objective of the present invention to provide pharmaceutical composition comprising about 450 mg of methylnaltrexone once daily as three tablets containing about 150 mg of methylnaltrexone for the treatment of OIC. Further, it is another objective of the present invention to provide pharmaceutical composition comprising about 150 mg of methylnaltrexone a day for the treatment of OIC.

It is also the objective of the present invention to develop a process for the preparation of the oral stable composition of methylnaltrexone or its pharmaceutically acceptable salt thereof, wherein, said process comprises; the initial sifting of methylnaltrexone bromide. Dissolve the docusate sodium in acetone and followed by addition of a weighed quantity of water to form binder solution. Granulate the sifted methylnaltrexone bromide with the prepared binder solution. Mix the granulating blend by adding sifted poloxamer, crospovidone, silicified microcrystalline cellulose. Prepare the Edetate calcium disodium solution by dissolving it in the purified water and added in the granulating blend for further granulation. Dry the granulated wet mass in rapid dryer till its LOD reaches below 3.0%. Sift the dried granules, and extragranular material i.e. silicified microcrystalline cellulose, crospovidone and croscarmellose sodium, colloidal silicon dioxide. Stearic acid is sifted and used to lubricate the blend, which is mixed for five minutes prior to compression and film coating.

It is another objective of the present invention to provide an alternate methylnaltrexone composition for an oral administration that is bioequivalent with the marketed Relistor® tablets.

### DETAILED DESCRIPTION OF INVENTION

The invention will now be described in detail in connection with certain preferred and optional aspects, so that various aspects thereof may be more fully understood and appreciated.

The present invention relates to an immediate release pharmaceutical compositions comprising methylnaltrexone or a pharmaceutically acceptable salt or derivative thereof, docusate and its pharmaceutically acceptable salt, and a pharmaceutically acceptable excipient.

In another embodiment, the pharmaceutical composition comprises about 150 mg of methylnaltrexone, or a salt thereof.

In certain embodiments, the compositions, and formulations thereof, comprise a salt of formula I:

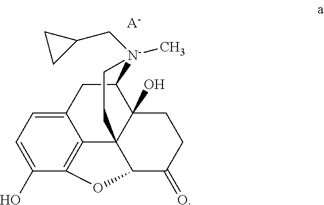

wherein A− is a suitable anion. In certain embodiments, A− is the anion of a Brønsted acid. Exemplary Brønsted acids include hydrogen halides, carboxylic acids, sulfonic acids, sulfuric acid, and phosphoric acid. In certain embodiments, A− is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A− is trifluoroacetate. In certain embodiments, A− is bromide.

In another embodiment, the invention provides pharmaceutical compositions containing from about 50 mg to about 500 mg of methylnaltrexone bromide. Preferably the invention provides pharmaceutical compositions containing from about 150 mg to about 450 mg of methylnaltrexone bromide equivalent to about 122.5 to about 367.5 mg methylnaltrexone.

In another embodiment, the composition comprises orally administering about 150 mg of methylnaltrexone, or a salt thereof. In a related embodiment, about 150 mg of methylnaltrexone is administered as one tablet comprising about 150 mg of methylnaltrexone.

In another embodiment, the composition comprises orally administering about 300 mg of methylnaltrexone, or a salt thereof. In a related embodiment, about 300 mg of methylnaltrexone is administered as two tablets each comprising about 150 mg of methylnaltrexone.

In another embodiment, the composition comprises orally administering about 450 mg of methylnaltrexone, or salt thereof. In one embodiment, about 450 mg of methylnaltrexone is administered as three tablets each comprising about 150 mg of methylnaltrexone.

In another embodiment, the invention provides a solid oral pharmaceutical composition containing 450 mg of methyl naltrexone bromide in a single unit dosage form.

In one embodiment the compositions of the present invention may be a coated or uncoated tablet, capsule, granule, powder.

In another embodiment, examples of suitable pharmaceutical dosage forms are included but not limited to granules, multiunit particulate systems (MUPS), pellets, spheres, tablets, dispersible tablets, soft capsules, hard capsules, mini-tablets, beads, particles. Preferable dosage forms are tablets or capsules.

In another embodiment, the present invention provides various dosage forms of methylnaltrexone or its salts, which include but are not limited to immediate release formulations, delayed release formulations, enteric coating, non-enteric coating, multiunit particulate system etc. Yet another embodiment of the present invention provides various processes to prepare oral pharmaceutical compositions of methylnaltrexone or its salts, and these processes include direct compression, wet granulation, dry granulation, spheronization, extrusion and spheronization spray drying or melt extrusion, etc. Additionally, compositions of the present invention can also be consumed as suspensions before processing into tablets or alternatively, fast disintegrating tablets can be added to water to form a fine suspension which can be consumed.

### Definitions

As used herein, the term “opioid induced constipation” (OIC) refers to a subject who suffers from constipation resulting from opioid therapy.

The term “constipation” as used herein, refers to a condition in which a subject suffers from infrequent bowel movements or bowel movements that are painful and/or hard to pass.

The term “about”, as used herein, is defined as all numerical values relating to amounts, weights, and the like, wherein each particular value is plus or minus 10%.

The term “therapeutically active agent” or “pharmaceutical active agent” or “drug” as used herein, refers to methylnaltrexone or its pharmaceutically acceptable salts thereof or it's a derivative thereof.

Methylnaltrexone chemically identified as (4R,4aS,7aR,12bS)-3-(cyclopropylmethyl)-4a,9-dihydroxy-3-methyl-2,4,5,6,7a,13-hexahydro-1H-4,12-methanobenzofuro[3,2-e]isoquinoline-3-ium-7-one. Its chemical structure is illustrated in Formula I.

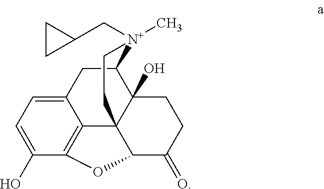 [SMILES: C[N+]1(CC2CC2)CC[C@]23c4c5ccc(O)c4O[C@H]2C(=O)CC[C@@]3(O)[C@H]1C5]

As used herein, the term “pharmaceutically acceptable salts thereof” refers to chemical structure illustrated in Formula II

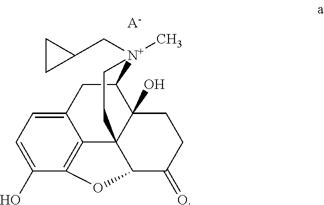

wherein A− is a suitable anion. In certain embodiments, A− is the anion of a Brønsted acid. Exemplary Brønsted acids include hydrogen halides, carboxylic acids, sulfonic acids, sulfuric acid, and phosphoric acid. In certain embodiments, A− is chloride, bromide, iodide, fluoride, sulfate, bisulfate, tartrate, nitrate, citrate, bitartrate, carbonate, phosphate, malate, maleate, fumarate sulfonate, methylsulfonate, formate, carboxylate, sulfate, methylsulfate or succinate salt. In certain embodiments, A− is trifluoroacetate. In certain embodiments, A− is bromide.

The term “pharmaceutically acceptable excipient” as used herein, is an inactive ingredient in a pharmaceutical composition and are well known to those skilled in the art for purpose of preparing methylnaltrexone composition for oral administration. Such pharmaceutically acceptable excipients, without limitation include, diluent or filler, disintegrant, chelating agent, wetting agent, surfactant, glidant, lubricant, coating agents.

The term “diluent” or “filler” is an excipient that adds bulkiness to a pharmaceutical composition.

The term “disintegrant” is an excipient that hydrates a pharmaceutical composition and aids in tablet dispersion.

The term “binder” is an excipient that imparts a pharmaceutical composition with enhanced cohesion or tensile strength.

The term “complexing agent or chelating agent” as used herein, means a molecule containing two or more electron donor atoms that can form coordinate bonds to a single metal ion. The term “chelating agent” is understood to include the chelating agent as well as salts thereof.

The term “wetting agent” as used herein is an excipient that imparts pharmaceutical compositions with enhanced solubility and/or wettability.

The term “glidant” as used herein is an excipient that imparts a pharmaceutical composition with enhanced flow properties.

The term “surfactant” as used herein, is used in its conventional sense in this invention. Any surfactant is suitable, whether it be amphoteric, non-ionic, cationic or anionic. Mixtures of surfactants are also suitable.

The term “lubricant” used herein, is a non-toxic excipient that decreases friction between tablet's surface and the die wall cavity in which the tablet was formed and to reduce wear and tear of dies and punches.

The term “coat” or “coating” or “coated” are equivalent terms and refers to film coating on to uncoated tablets or granules or powder.

The term “enteric coating” or “enteric coat” refers to no release of active pharmaceutical ingredient at highly acidic pH such as pH 1.2 using USP II apparatus in 900 mL water or 0.1N HCL at 50 or 100 rpm at temperature 37° C.±0.5° C.; or a coat that is stable at the highly acidic pH found in the stomach; and contains at least 10% enteric coating polymer.

The terms “non-enteric polymer” and “pH independent polymer” are here understood to refer to a polymer which is non-enteric, i.e., which is not more soluble in non-acidic media than in acidic media. The terms “non-enteric polymer” and “pH independent polymer” therefore encompass polymers which are equally soluble in acidic, and neutral or basic media. The terms “non-enteric polymer” and “pH independent polymer” may additionally encompass polymers which are more soluble in acidic media than in neutral or basic media and/or swellable in non-acidic media.

The term “partition coefficient” as used herein, refers to the ratio of solubility of the substance in an n-octanol to solubility of the substance in water.

The term “log P” as used herein, refers to the partition coefficient of a substance. The log P of a substance is the base ten logarithm of the ratio of solubility of the substance in an n-octanol to solubility of the substance in water.

The term “immediate release” (IR) means, for example, a release of at least 60% of the drug under physiological conditions (pH, temperature), such as within 60 minutes or less, such as within 30 or less, or within 20 minutes or less, or within 15 minutes or less. An immediate release drug product is considered rapidly dissolving when no less than 85% of the drug substance dissolves within 30 minutes in a volume of 900 ml or less in media of varying pH.

The term “bioequivalent” means the absence of a significant difference in the rate and extent to which the active ingredient or active moiety in pharmaceutical equivalents or pharmaceutical alternatives becomes available at the site of drug action administered at the same molar dose under similar conditions in an appropriately designed study. In practice, two products are considered bioequivalent if the 90% confidence interval of the Cmax, AUC, or, optionally, Tₘₐₓ is within the range of 80.00% to 125.00%.

The term “subject”, as used herein, means a mammal and includes human and animal subjects, such as domesticated animals (e.g., horses, dogs, cats, etc.) and experimental animals (e.g., mice, rats, dogs, chimpanzees, apes, etc.).

The terms “treat” or “treating,” as used herein, refers to partially or completely alleviating, inhibiting, delaying onset of, reducing the incidence of, ameliorating and/or relieving a disorder or condition, or one or more symptoms of the disorder, disease or condition.

In another embodiment, the examples of diluent or filler include but not limited to calcium phosphate, dicalcium phosphate, tricalcium phosphate, calcium sulfate, anhydrous lactose, spray dried lactose, hydrated lactose, cellulose, spray dried microcrystalline cellulose, spray dried combinations comprising microcrystalline cellulose and lactose, silicified microcrystalline cellulose, kaolin, bentonite, mannitol, starch, magnesium carbonate, sorbitol, sucrose, inositol, compressible sugar, trehalose and xylitol, and mixtures thereof.

In another embodiment, the composition comprises from 0 to about 90%, or from about 15 to about 85%, or from about 20 to about 75%, or from about 30 to about 70%, or from about 15 to about 30%, or from about 50 to about 90%, or from about 60 to about 85%, of diluent or filler.

In another embodiment, the examples of disintegrants include but are not limited to crospovidone, sodium croscarmellose and/or sodium starch glycolate, pregelatinized starch, low substituted hydroxypropyl cellulose (L-HPC).

In another embodiment, the composition may comprise disintegrant from 0 to about 20%, or from about 5 to about 20%, or from about 5 to about 15% by weight of composition.

In another embodiment, the examples of chelating agents include but not limited to ethylenediaminetetraacetic acid (also synonymous with EDTA, edetic acid, versene acid, and sequestrene), and EDTA derivatives, such as sodium EDTA, and potassium EDTA, diammonium EDTA, dipotassium EDTA, disodium EDTA, TEA-EDTA, tetrasodium EDTA, tripotassium EDTA, trisodium EDTA, HEDTA, and trisodium HEDTA, and related salts thereof. Other chelating agents include niacinamide and derivatives thereof and sodium desoxycholate and derivatives thereof, ethylene glycol-bis-(2-aminoethyl)-N,N,N′,N′-tetraacetic acid (EGTA) and derivatives thereof, diethylenetriaminepentaacetic acid (DTPA) and derivatives thereof, N,N-bis(carboxymethyl)glycine (NTA) and derivatives thereof, nitrilotriacetic acid and derivatives thereof. Still other chelating agents include citric acid and derivatives thereof. Citric acid also is known as citric acid monohydrate. Derivatives of citric acid include anhydrous citric acid and trisodiumcitrate-dihydrate. In some embodiments, chelating agent is selected from EDTA or an EDTA derivative or EGTA or an EGTA derivative. In some embodiments chelating agent is EDTA disodium such as, for example, EDTA disodium hydrate.

Common calcium salt chelating agents include, but are not limited to calcium ethylenediaminetetra acetic acid (EDTA) and calcium salt EDTA derivatives, calcium ethylene glycol-bis-(2-aminoethyl)-N,N,N′,N′-tetraacetic acid (EGTA) and calcium salt EGTA derivatives, calcium diethylenetriaminepentaacetic acid (DTPA) and calcium salt DTPA derivatives, calcium N,N-bis(carboxymethyl)glycine (NTA) and calcium salt NTA derivatives, and calcium citrate and derivatives thereof In some embodiments, chelating agent is selected from calcium EDTA or a calcium salt EDTA derivative or calcium EGTA or a calcium salt EGTA derivative. In some embodiments chelating agent is calcium EDTA disodium such as, for example, calcium EDTA disodium hydrate.

In another embodiment, the composition may comprise chelating agent from 0 to about 5%, or from about 0.01 to about 2%, or from about 0.01 to about 1% by weight of composition.

In another embodiment, the examples of wetting agents include but not limited to poloxamer, polyoxyethylene ethers, polyoxyethylene sorbitan fatty acid esters polyoxyethylene fatty acid esters, polyethylene glycol fatty acid esters, polyoxyethylene hydrogenated castor oil, polyoxyethylene alkyl ether, polysorbates, such as polysorbate 80, cetyl alcohol, glycerol fatty acid esters (e.g., triacetin, glycerol monostearate, and the like), polyoxymethylene stearate, sorbitan fatty acid esters, sucrose fatty acid esters, benzalkonium chloride, polyethoxylated castor oil, and the like, and combinations thereof.

In another embodiment, the composition comprises wetting agent from about 0 to 10%, or from about 0.1% to about 5%, or from about 0.1% to about 3% by weight of the composition.

In another embodiment, the examples of surfactants include but not limited to cetostearyl alcohol, cetomacrogol emulsifying wax, gelatin, casein, docusate sodium, benzalkonium chloride, calcium stearate, polyethylene glycols, phosphates, polyoxyethylene sorbitan fatty acid esters (e.g. Polysorbate 80, Polysorbate 20), gum acacia, cholesterol, tragacanth, polyoxyethylene 20 stearyl ethers, polyoxyethylene alkyl ethers, polyoxyethylene castor oil derivatives, pegylated hydrogenated castor oils, sorbitan esters of fatty acids, Vitamin E or tocopherol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids (i.e. glycerol monostearate), ethylene glycol palmitostearate, polyoxylglycerides, propylene glycol monocaprylate, propylene glycol monolaurate, alkyl aryl polyether alcohols (Triton®) and polyglyceryl oleate.

In another embodiment, the composition comprises surfactant in an amount from about 0 to 30%, or from about 0.1 wt % to about 15 wt %, or from about 0.1 wt % to about 10% by weight of the composition.

In another embodiment, the examples of glidant include but not limited to colloidal silicon dioxide, calcium phosphate tribasic, magnesium silicate, and talc, or combinations thereof.

In another embodiment, the composition may comprise glidant in an amount from about 0.1 to about 5% by weight of the composition.

In another embodiment, the examples of binder include but not limited to microcrystalline cellulose, silicified microcrystalline cellulose, gelatin, sugars, polyethylene glycol, natural and synthetic gums, polyvinylpyrrolidone, pregelatinized starch, hydroxypropyl cellulose, and hydroxypropyl methylcellulose, or combinations thereof.

In another embodiment, the composition may comprise of a binder from about 0 wt % to about 25 wt %, or from about 0.1 wt % to about 25 wt % of the composition.

In another embodiment, the examples of coating excipients include but are not limited to those known in the art, such as cellulose derivatives (hydroxypropyl methylcellulose, hydroxyethylcellulose, hydroxypropyl cellulose and their derivatives), acrylic and methacrylic copolymers of different molecular weights, and mixtures thereof, water-soluble polymers for instance, aminoalkyl methacrylate copolymer E, hypromellose, methyl cellulose, methyl hydroxyethyl cellulose, Opadry, calcium carmellose, sodium carmellose, polyvinyl pyrrolidone, polyvinyl alcohol, dextrin, pullulan, gelatin, agar and gum Arabic, among others. Preferably coating composition comprises polyvinyl alcohol, titanium dioxide, talc and polyethylene glycol. The coating layers over the tablet may be applied as solution/dispersion of coating ingredients using conventional techniques known in the art selected from spray coating in a conventional coating pan or fluidized bed processor, dip coating, and the like. In one embodiment, weight gain after coating is up to about 5%, preferably up to about 3%.

In one embodiment, the examples of lubricant include but are not limited to magnesium stearate, stearic acid, palmitic acid, calcium stearate, talc, carnauba wax and sodium stearyl fumarate. Preferably, the lubricant of the present invention is present in an amount of about 0.01 to about 5% by weight, relative to the weight of the solid formulation.

In another embodiment the composition of the present invention also may include a rapid-acting disintegrant, wherein the composition dissolves within about 15 minutes in the stomach. In at least one embodiment, at least 50% of the methylnaltrexone in the composition is dissolved in 15 minutes.

In another embodiment, at least 60%, 75%, 80%, 85%, 90%, 95%, or even 99% of the methylnaltrexone in the present composition is dissolved in 15 minutes.

In another embodiment, the methylnaltrexone in the present composition can dissolve within 10 minutes or even within 5 minutes.

Yet in another embodiment, the dissolution of the present compositions may be simulated by in vitro studies in a dissolution apparatus with paddles at 100 rpm in 900 ml 0.1 N HCl at 37° C.±0.5° C.

Yet in another embodiment, the dissolution of the present compositions may be simulated by in vitro studies in a dissolution apparatus with paddles at 50 rpm in 1000 ml 0.1 N HCl at 37° C.±0.5° C.

Yet in certain embodiment, the dissolution of the present composition may be simulated by in vitro studies in dissolution apparatus with USP-I (basket) at 100 rpm in 500 mL 0.1N HCl at 37° C.±0.5° C.

In another embodiment, the present invention provides a composition comprising methylnaltrexone and a pharmaceutically acceptable excipient, wherein the composition in solution yields an octanol/water partition coefficient for methylnaltrexone of less than 0.25 under acidic conditions, in certain embodiments at a pH between 2 and 4. A pH of between 2 and 4 is used to simulate the physiological conditions of the stomach. In some embodiments, such compositions are formulated for oral administration. In some embodiments, a composition for oral administration is formulated into a tablet. The partition coefficient of a compound may be determined by procedures known in the art, for example, in the U.S. Pharmacopeia.

In another embodiment, the present invention provides a process for preparation of methylnaltrexone composition for oral administration, wherein, said process comprises; (i) Sift methylnaltrexone bromide; (ii) Dissolve the docusate sodium USP in weighed qty. of acetone USP with continuous stirring; (iii) Add weighed quantity of purified water in step (ii); (iv) Granulate the blend of Step (i) with binder solution of step (iii) using suitable granulation parameters; (v) Add sifted poloxamer, crospovidone in step (iv) and mix it; (vi) Load sifted silicified microcrystalline cellulose in step (v) and mix it; (vii) Weigh the required quantity of edetate calcium disodium and dissolve it in sufficient quantity of purified water; (viii) Granulate the step (vi) using step (vii) solution. (ix) unload the wet mass of step (viii) and dry it in rapid dryer till LOD reaches below 3.0% w/w (x) sift and mixt the dried granules and extragranular material silicified microcrystalline cellulose, crospovidone and croscarmellose sodium; (xi) Sift the colloidal silicon dioxide and mix it with prelubricated blend step (x); (xii) sift extragranular stearic acid and lubricates the blend of step (xi); (xiii) compress the blend of (xii) with suitable tooling for tablets followed by coating.

The following examples are meant to illustrate the present invention and should not be construed as limiting its scope.

### EXAMPLES

### Example 1: Methylnaltrexone Solid Oral Composition

TABLE 1
Methylnaltrexone bromide tablets 150 mg

| Sr. | | Quantity per Tablet in | |
|---|---|---|---|
| No | Name of the ingredient | 150 mg | % w/w |
| | Intragranular | | |
| 1 | Methylnaltrexone Bromide | 150.000 | 28.11 |
| 2 | Docusate Sodium | 16.000 | 3.00 |
| 3 | Poloxamer | 10.700 | 2.01 |
| 4 | Edetate Calcium Disodium | 1.290 | 0.24 |
| 5 | Crospovidone | 9.500 | 1.78 |
| 6 | Silicified Microcrystalline Cellulose | 90.000 | 16.87 |
| | Acetone | Q.S. | — |
| | Purified Water | Q.S. | — |
| | Extragranular | | |
| 7 | Silicified Microcrystalline Cellulose | 195.310 | 36.61 |
| 8 | Crospovidone | 15.500 | 2.91 |
| 9 | Croscarmellose Sodium | 25.900 | 4.85 |
| 10 | Colloidal Silicon Dioxide | 2.500 | 0.47 |
| 11 | Stearic Acid | 1.300 | 0.24 |
| | Core Tablet Weight (mg) | 518.000 | 97.09 |
| | Film Coating | | |
| 12 | Opadry (white) | 15.540 | 2.91 |
| 13 | Purified Water | Q.S. | — |
| | Coated Tablet Weight (mg) | 533.540 | 100.00 |

### Example 2: Manufacturing Process for Methylnaltrexone Tablets 150 mg (Example 1)

1. Check and verify the weights of dispensed ingredients.
2. Sift methylnaltrexone bromide.
3. Dissolve the docusate sodium in weighed quantity of acetone with continuous stirring.
4. Add weighed quantity of purified water in Step-3.
5. Granulate the blend of Step 2 with binder solution of step 4. using suitable granulation parameters.
6. Add sifted poloxamer, crospovidone in step 5, and mix it.
7. Load sifted silicified microcrystalline cellulose in step 6 and mix it.
8. Weigh the required quantity of edetate calcium disodium and dissolve it in sufficient quantity of purified water.
9. Granulate the Step 7 material using step 8 solution.
10. Unload the wet mass of step 9 and dry it in rapid dryer till its LOD reaches below 3.0% w/w.
11. Sift the dried granules of step 10.
12. Sift extragranular material i.e. silicified microcrystalline cellulose, crospovidone and croscarmellose sodium and mix it with step 11 granules.
13. Sift colloidal silicon dioxide and mix it with step 12 prelubricated blend.
14. Sift extra granular stearic acid.
15. Lubricate the blend of step 13 using step 14 sifted material and mix it.
16. Compress the blend of step 15 using suitable tooling into tablet.
17. Disperse the Opadry white in purified water with continuous stirring.
18. Coat the tablet of step 16 using opadry dispersion of step 17 using suitable coating parameters with weight buildup 3.0% w/w.

### Example 3: Partition Coefficient Determination of Methylnaltrexone Composition (Example 1)

The partition coefficient of methylnaltrexone composition in octanol-water was evaluated. A one tablet was dissolved in 75 mL of 1-octanol, which had been saturated with water. Following this, 75 mL of water saturated with 1-octanol was added. The mixture was then shaken overnight (16 hours) at room temperature. Afterward, 1 mL of the 1-octanol phase was diluted to 4 mL with the diluent, while 1 mL of the aqueous phase was diluted to 20 mL with the same diluent. The analysis was conducted via HPLC to determine the partition coefficient of the methylnaltrexone composition. The octanol-water partition coefficient at pH 2 and 4 was found to be in the range of about 0.05 to less than 0.25. Respective pH achieved by adjusting the pH of water using hydrochloric acid.

### Example 4: Dissolution Study of Methylnaltrexone Bromide Tablets 150 mg (Example 1)

Dissolution Conditions: Dissolution apparatus with USP-I (basket) at 100 rpm in 500 mL of 0.1N HCl at 37° C.±0.5° C.

TABLE 2
% in vitro release of methylnaltrexone using apparatus USP-I (basket) at
100 rpm in 500 mL of 0.1N HCl at 37° C. ± 0.5° C.

| Time (min) | In vitro release (%) |
|---|---|
| 5 | 96 |
| 10 | 98 |
| 15 | 99 |
| 20 | 99 |
| 30 | 99 |
| 45 | 99 |
| 60 | 99 |

### Example 5: Stability Studies

Tablet composition in accordance with example 1 containing 150 mg of methylnaltrexone bromide, packed in 50 cc HDPE bottle with 1 gm silica gel. The pack was subjected to storage stability condition such as 25° C./60% RH for three months. The pack was also subjected to accelerated storage conditions of 40° C./75% RH for a period of up to six months. The tablets were analyzed for water content, in-vitro dissolution, related substances or degradation products and assay.

TABLE 3
In vitro release, % assay, water content, and degradation impurity of 150
mg of methylnaltrexone tablets of example 1 packaged in 50 cc HDPE bottle
with 1 gm silica gel after 1, 3, and 6 month accelerated stability condition
of 40° C./75% RH and 3-month stability condition of 25° C./60% RH.

| | Storage Condition | | | | | |
|---|---|---|---|---|---|---|
| | NA | 40° C./75 | 25° C./60 | 40° C./75 | 40° C./7 | |
| | | % RH | % RH | % RH | 5% RH | |
| Tests | Initial | 1 M | 3 M | 3 M | 6 M | Limit |
| Water content by | 3.45 | 3.31 | 3.54 | 3.49 | 3.48 | NMT 7.0 |
| KF (% w/w) | | | | | | |

Dissolution: % in vitro release of methylnaltrexone in 0.1N HCl,
500 mL, USP Type-I (Basket), 100 RPM at temp. 37° C. ± 0.5° C.

| Time | Initial | 1 M | 3 M | 3 M | 6 M | Limit |
|---|---|---|---|---|---|---|
| 5 Min | 96 | 99 | 97 | 98 | 97 | NLT 80% (Q) |
| 10 Min | 98 | 98 | 99 | 99 | 99 | of the labelled |
| 15 Min | 99 | 96 | 100 | 100 | 99 | amount of |
| 20 Min | 99 | 95 | 100 | 100 | 99 | methylnaltrexone |
| 30 Min | 99 | 93 | 100 | 100 | 100 | dissolved in 60 |
| 45 Min | 99 | 92 | 101 | 100 | 100 | minutes |
| 60 Min | 99 | 90 | 101 | 100 | 100 | |

Assay (%)

| Each Tablet Contains | | | | | | |
|---|---|---|---|---|---|---|
| Methylnaltrexone | Initial | 1 M | 3 M | 3 M | 6 M | Limit |
| mg/Tablet | 149.71 | 146.70 | 150.29 | 150.36 | 150.53 | 135.00 mg to |
| | | | | | | 165.00 mg |
| % Label Claim | 99.8 | 97.8 | 100.2 | 100.2 | 100.4 | 90.0 to 110.0% |
| | | | | | | of label claim |

Degradation Product by HPLC (%)

| — | Initial | 1 M | 3 M | 3 M | 6 M | Limit |
|---|---|---|---|---|---|---|
| S- Methylnaltrexone | BDL | BDL | BDL | BDL | ND | NMT 0.2% |
| | (0.02) | (0.015) | (0.02) | (0.02) | | |
| Naltrexone | ND | ND | BDL | ND | ND | NMT 0.2% |
| | | | (0.02) | | | |
| N-Butenyl | BDL | BDL | BDL | BDL | BDL | NMT 0.2% |
| oxymorphone | (0.01) | (0.005) | (0.02) | (0.01) | (0.009) | |
| Acetylmethylnaltrexone | BDL | ND | ND | BDL | ND | NMT 0.2% |
| | (0.01) | | | (0.01) | | |
| Acetylnaltrexone | ND | ND | ND | ND | ND | NMT 0.2% |
| O-Methyl | 0.05 | BQL | 0.06 | 0.05 | 0.043 | NMT 0.2% |
| methylnaltrexone | | (0.036) | | | | |
| Any Unspecified | 0.07 | 0.228 | 0.06 | 0.06 | 0.027 | NMT 0.2% |
| Degradation Product | (RRT | (RRT | (RRT | (RRT | (RRT | |
| | 1.09) | 2.03) | 0.80) | 0.73) | 3.06) | |
| Total Degradation | 0.26 | 0.47 | 0.19 | 0.25 | 0.112 | NMT 1.0% |
| Products | | | | | | |

ND = Not Detected, BDL: Below detection Limit, BQL: Below Quantification Limit

### Example 6: Bioequivalence Study

Study Design: Oral Relative Bioavailability Study Comparing methylnaltrexone bromide tablets 150 mg (Test Product; Example 1), with RELISTOR® (methylnaltrexone bromide tablets 150 mg), (Dose: 1*3 tablets).

TABLE 4
Bioequivalence study

| | | | | | | | Intra- |
|---|---|---|---|---|---|---|---|
| | | | | | T/R | 90% | subject |
| | | | Geomean | Geomean | Ratio | Confidence | CV |
| Comparison | N | Parameter | Test | Reference | (%) | Interval | (%) |
| Relistor ® | 23 | Ln(AUC₀₋ₜ) | 291.7197 | 306.4711 | 95.19 | [83.85, | 26.21 |
| 150 mg | | | | | | 108.05] | |
| (3 tablets) | | Ln(AUC_{0-inf}) | 296.5963 | 313.5384 | 94.60 | [83.42, | 25.99 |
| Vs | | | | | | 107.27] | |
| Test Product | | | | | | | |
| (3 tablets) | | | | | | | |

TABLE 5
Time to reach maximum plasma concentration (Tₘₐₓ)

| | | |
|---|---|---|
| Tₘₐₓ(Hr) | Reference Product (R) | 1.50 (0.50-4.25) |
| Median (Min-Max) | Test Product (T) | 1.75 (0.75-4.50) |

1. A stable pharmaceutical composition comprising: (i) methylnaltrexone or a pharmaceutically acceptable salt thereof; (ii) docusate or a pharmaceutically acceptable salt thereof; and (iii) one or more pharmaceutically acceptable excipients.

2. The composition according to claim 1, wherein the composition is suitable for oral administration.

3. The composition according to claim 1, wherein the composition comprises from about 50 mg to about 500 mg, or preferably from about 150 mg to about 450 mg, of methylnaltrexone bromide.

4. The composition according to claim 1, wherein the composition is administered for the treatment of opioid-induced constipation in adult patients with cancer or chronic non-cancer pain.

5. The composition according to claim 1, wherein the composition is administered at a dose of 150 mg or 450 mg once daily for the treatment of opioid-induced constipation in adult patients with cancer or chronic non-cancer pain.

6. The composition according to claim 1, wherein the composition is in a solid dosage form.

7. The composition according to claim 6, wherein the solid dosage form is selected from tablets, capsules, dispersible tablets, chewable tablets, granules, spheres, powders, multiunit particulate systems (MUPS), beads, and particles.

8. The composition according to claim 1, wherein the composition comprises at least one or more excipients selected from binders, chelating agents, wetting agents, lubricants, disintegrants, glidants, non-functional coating agents, surfactants, and combinations thereof.

9. The composition according to claim 1, wherein the composition is an immediate-release composition.

10. The composition according to claim 1, wherein the composition releases about 60%, about 75%, about 80%, about 85%, about 90%, about 95%, or about 99% of the methylnaltrexone within 15 minutes when measured using USP Type I (basket) apparatus at 100 rpm in 500 mL of 0.1 N HCl at 37° C.±0.5° C.

11. A pharmaceutical composition for oral administration comprising a solid dosage form of (i) methylnaltrexone or a pharmaceutically acceptable salt thereof, and (ii) docusate or a pharmaceutically acceptable salt thereof, wherein the composition in solution has an apparent octanol/water partition coefficient for methylnaltrexone of less than 0.25 at a pH between 2 and 4.

12. The composition according to claim 11, wherein the composition in solution has an apparent octanol/water partition coefficient for methylnaltrexone in the range of about 0.05 to less than 0.25 at a pH between 2 and 4.

13. An immediate-release methylnaltrexone composition according to claim 11, wherein the composition is bioequivalent to RELISTOR® 150 mg tablets.
